(19) United States
(12) Patent Application Publication
(10) Pub. No.: US 2002/0188219 A1
(43) Pub. Date: Dec. 12, 2002

(54) Method and apparatus for inferring physical/mental fitness through eye response analysis

(76) Inventor: Eytan Suchard, Kiryat Bialik (IL)

Correspondence Address: G.E. EHRLICH (1995) LTD., c/o ANTHONY CASTORINA, SUITE 207, 2001 JEFFERSON DAVIS HIGHWAY, ARLINGTON, VA, 22202, US

(21) Appl. No.: 10/158,890

(22) Filed: Jun. 3, 2002

(30) Foreign Application Priority Data
Jun. 6, 2001 (IL) 143597

(51) Int. Cl. A61B 13/00

(52) U.S. Cl. 600/558

(57) ABSTRACT

A method and apparatus are disclosed for testing the physical/mental fitness of a subject by observing his/her eye responses to stimuli in the form of images in motion displayed on a screen. The method comprises the steps of monitoring the eye behavior of the subject in response to movement of the images, analyzing the resulted responses, comparing the results to a reference eye pattern, and determining the level of fitness according to the comparison results. The apparatus is intended to be used for self-testing e.g. by bus drivers or equipment operators and may be coupled so as to disable operation of the vehicle or equipment by unfit subjects.

### BACKGROUND OF THE INVENTION

The present invention generally relates to apparatus and method for testing the level of physical and/or mental fitness of individuals. More particularly, the invention is directed to testing individual fitness by analyzing his/her eye behavior pattern.

It is well known that the major cause of car accidents is driving under the influence of alcohol or other drugs, or suffering under temporary or permanent fatigue or illness. Obviously, the risk factor is even greater in the case of buses or heavy trucks drivers. Moreover, the problem of physical/mental impairment is not limited to the field of transportation, as it has been recognized that there are many other fields, such as the operation of production line machines or heavy equipment, where such impairment places the operator and others at higher risks of injury or even fatal accidents.

In order to avoid such risks there have already been proposed certain testing devices for ascertaining the physical/mental condition or at least alerting individuals of their unfitness.

These devices were based on detecting parameters such as decrease of eye-hand coordination, cognitive skills or short-term memory. However, these methods were found unsatisfactory, being time consuming and involving the services of skilled personnel.

Other methods were developed, based on eye reactions to stimuli, known as pupilometers, which provided indications of subjects' autonomic nervous system by measuring the pupil response to variable light stimuli. It has been found that such pupilometers are useful in detecting fatigue, the presence of alcohol and drugs, Alzheimer's disease symptoms as well as other conditions directly related to the nervous system functional impairment. However, pupilometers still enjoyed only limited acceptance, particularly in the field of transportation, due in part to certain deficiencies of identifying user's fitness under various conditions.

A further development in this direction is disclosed in the recent Publication No. WO99/18842. The device described has been designed to observe and automatically record the eye movements by using an illumination source, a camera and data processor for measuring eye gaze positions. The eye tracker device generated a continuous stream of data representing eye gaze positions at sequential time intervals.

In more detail, when staring at a moving object, the human eye undergoes extremely fast rotations (up to 600 degrees per second), called “saccadic eye movements”, or saccades. It is requested that the eye tracker device be capable of following and tracking the eye even though moving at the very high speeds of saccadic eye movements. Analyzing the data reflecting these movements revealed a series of eye fixations, separated by sudden jumps in-between the fixations. Also measured were characteristics of the saccades, mainly the saccadic velocity from which important information can be derived regarding the physical/mental condition of the inspected individual.

While this latest described device seems to be a development in the right direction, there are still certain disadvantages involved mainly regarding the implementation thereof as a readily useful, “do it yourself” test instrument.

It is thus the prime object of the invention to provide an eye detector device of the kind referred to which is self-contained and easy to operate, rendering it to be used as a “test yourself” instrument.

It is a still further object of the invention to include additional variants that will improve the reliability of the test and shorten the duration thereof.

It is a further object of the invention to apply more than one moving image within the field of sight along with variation in the relative speeds, directions, colors, sizes and shapes so that an increased amount of information can be derived at less testing time.

It is a still further object of the invention to couple the device to the “dangerous” machine as above defined thus avoiding operation thereof should the results of the test so dictate.

### SUMMARY OF THE INVENTION

Thus provided according to one aspect of the invention is a method of testing the physical/mental fitness of a subject by observing his/her eye responses to stimuli in the form of images in motion displayed on a screen, comprising the steps of monitoring the eye behavior of the subject in response to movements of the image, analyzing the resulted responses, comparing the results to normally expected eye behavior pattern and determining the level of fitness according to the comparison results.

According to another aspect of the present invention there is provided an apparatus for testing the physical/mental fitness of a subject by observing his/her eyes responses to stimuli in the form of images in motion displayed on a screen. The apparatus comprises a housing, an eye piece, a screen, means for displaying on the screen one or more images in movement along a pre-determined track, means for viewing and recording the eye behavior of the subject, means for storing eye behavior of a reference subject and means for comparing the eye behavior of the subject and reference subject.

### BRIEF DESCRIPTION OF THE DRAWINGS

These and further features and advantages of the invention will become more clearly understood in the light of the ensuing description of a preferred embodiment thereof, given by way of example only, with reference to the accompanying drawings, wherein

FIG. 1 is a schematic representation of a testing device designed according to the principles of the present invention;

FIG. 2 is a schematic enlarged representation of an LCD screen and of an example of displayed images.

FIG. 3 illustrates a first of a hierarchy of recommended tests according to a preferred embodiment of the invention;

FIG. 4 illustrates a second test; and

FIG. 5 illustrates a third test.

### DETAILED DESCRIPTION OF THE PREFERRED EMBODIMENTS

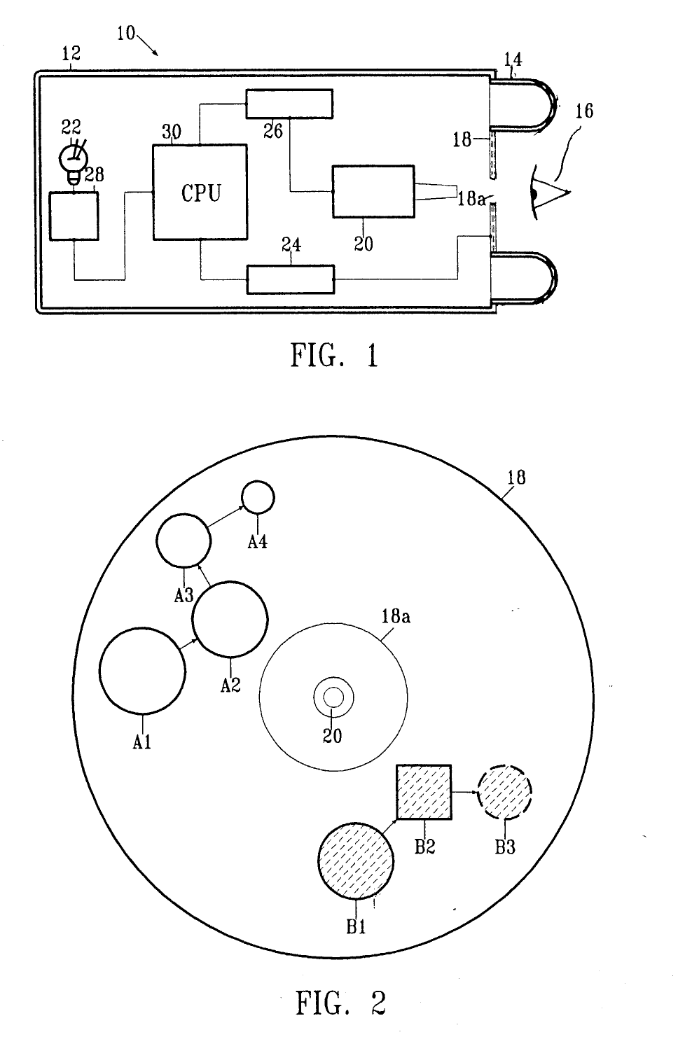

Referring to FIG. 1 of the drawings, it will be seen that testing device 10 comprises a housing 12 say of a cylindrical shape, and eye rest pad 14 for the eye 16 of the tested subject. LCD screen 18 is positioned as shown, having a central opening 18a.

A serial or CCD camera 20 is positioned in front of the opening 18a for viewing and recording the movements of the eye 16.

A light source 22 is installed at the rear side of the housing 12. So far for the “hardware” aspect of the testing device 10.

From the “software” aspect there are provided: Generator 24 which controls the animation of the moving image targets displayed on the screen 18 such as A and B as exemplified in FIG. 2.; camera data processing unit 26; lighting operating unit 28; and central processing unit 30 for compiling and analyzing the output of the units 24, 26 and 28, namely presenting the results of the test (see below).

The routine of the tests conducted is of vital importance to the effectiveness of the method, since it is based on recent brain analysis and research, according to which

“The inferior temporal centers in the lower ventral system seem to be involved in identifying objects whereas the parietal centers in the upper (dorsal) system seem to be involved in locating objects” (Vision Science, Photons to Phenomenoloy, Stephen E. Palmer, ISBN 0-262-16183-4, page 38.)

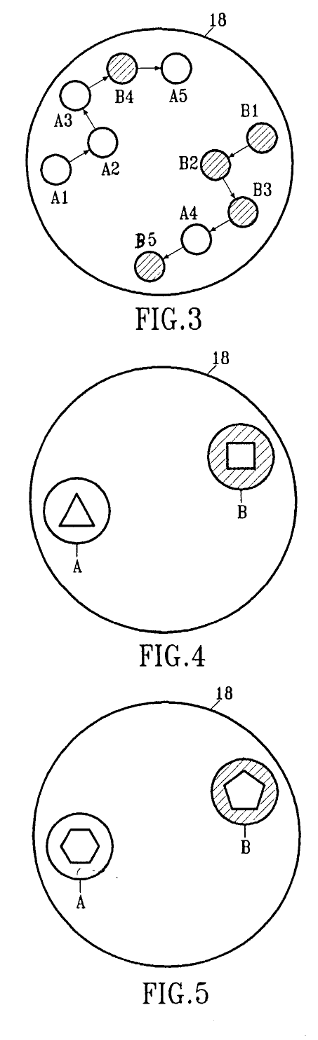

Hence, the tests are designed in a hierarchic order and involving both color and shape recognition parameters, as will be now exemplified with reference to FIGS. 3-5.

Test No. 1 (FIG. 3)

There will appear on the screen 18 a first image A, say in red color, and a second, green image B.

For a while the images A and B move (governed by the unit 24) to change their positions along a random path (A1→A2→A3; B1→B2→B3).

The subject will be instructed to follow only the movement of the red images A. The camera 20 will trace and monitor the eye behavior (pupil, iris and/or retina) during the time of movement. The data gathered during this phase of the test is continuously stored and analyzed by CPU 30.

At the next stage, the positions of certain images are switched so that, for example, A4 appears on the path of images B and B4—on the path of images A.

Then, another exchange of positions takes place—see A5 and B5, and so on.

It will take some time for the subject to shift and re-gain focus on the images A4 and A5, which time lag is measured and recorded.

Test No. 2 (FIG. 4)

The test is conducted in a similar manner only that now two different geometrical shapes are used: a triangle for images A and a square for images B.

Test No. 3 (FIG. 5)

It is similar to Test 2., but the geometrical shapes more closely resemble each other such as a pentagon and a hexagon, respectively.

It will be readily understood that the above tests are illustrative, given only as guidelines for correctly programming a variety of others, perhaps more sophisticated and diversified ones.

According to another proposed variation, the light source 22 may be switched on and off during any of the tests. This will add to the test findings the pupil contraction reactions speed and intensity and yield additional data relating to the subject's mental fitness.

Finally, and according to an important aspect of the present invention, there will be established (empirically or otherwise) a “standard” or scale of results, stored in the CPU 30. The analytical or scientific basis of the results is of no importance but merely the deviations or differential irregularities of the results, which will clearly and unmistakably prove that the subject of the test is in a disqualified condition for the purposes in question, regardless the reasons that brought him/her to such condition.

In such case, there will be emitted any kind of a warning signal, marking that the subject should refrain from “dangerous” activity, e.g. driving a motorcar.

In a more sophisticated level, the device can be provided with transmitting means (R.F., I.R. Blue-Tooth etc.) for immobilizing the motorcar, thus disabling driving by the disqualified subject.

Those skilled in the art to which this invention pertains will appreciate that numerous changes, variations or modifications are readily applicable thereto without departing from the scope of the invention as defined in and by the appended claims.

What is claimed is:

1. A method of testing the physical/mental fitness of a subject by observing his/her eye responses to stimuli in the form of images in motion displayed on a screen, comprising the steps of:
(i) monitoring the eye behavior of the subject in response to movements of the image;
(ii) analyzing the resulted responses;
(iii) comparing the results to normally expected eye behavior pattern; and
(iv) determining the level of fitness according to the comparison results.

2. The method of claim 1 further comprising the step of displaying simultaneously two or more images of different color and/or shape moving along different trails.

3. The method of claim 2 further comprising the step of switching positions between different images during movement thereof along their respective tracks.

4. The method of claim 3 wherein the images are circular.

5. The method of claim 3 wherein the images are polygonal.

6. The method of claim 3 wherein the images are encircled polygonal.

7. The method of claim 3 wherein a background light is activated during the movement of the images.

8. An apparatus for testing the physical/mental fitness of a subject by observing his/her eyes responses to stimuli in the form of images in motion displayed on a screen, comprising:
a housing;
an eye piece;
a screen;
means for displaying on the screen one or more images in movement along a pre-determined track;
means for viewing and recording the eye behavior of the subject;
means for storing eye behavior of a reference subject; and
means for comparing the eye behavior of the subject and reference subject.

9. The apparatus of claim 8 further comprising a background light source.

10. The apparatus of claim 8 wherein the screen is an LCD screen having a central opening through which the subject's eye is viewed by a serial camera.

11. The apparatus of claim 10 further comprising means for selecting the shape/color/size of said image(s).

12. The apparatus of claim 11 wherein a pair of different images are displayed, means being provided for selectively switching the positions thereof while moving along their respective tracks.

13. The apparatus of claim 12 further comprising means for disabling the operation of a remotely located device, such as a motor of a vehicle, pending the results of said comparing means.
